(19) United States
(12) Patent Application Publication
(10) Pub. No.: US 2016/0145459 A1
(43) Pub. Date: May 26, 2016

(54) PROCESS FOR PRODUCING A MICROCAPSULE DISPERSION COMPRISING MICROCAPSULES WITH A HYDROPHILIC CAPSULE CORE

(71) Applicant: BASF SE, Ludwigshafen (DE)

(72) Inventors: Regina KLEIN, Speyer (DE); Tina SCHRÖDER-GRIMONPONT, Jockgrim (DE); Patrick LIEBACH, Landau (DE); Britta KATZ, Dannstadt-Schauernheim (DE); Peter HAHN, Obrigheim (DE); Achim PIESCH, Edingen-Neckarhausen (DE); Jutta BRUST, Kallstadt (DE); Joseph STRACKE, Heuchelheim (DE)

(21) Appl. No.: 14/897,986

(22) PCT Filed: May 27, 2014
(86) PCT No.: PCT/EP2014/060945

§ 371 Date: Dec. 11, 2015

(30) Foreign Application Priority Data
Jun. 14, 2013 (EP) 13172032.8

(51) Int. Cl. C09D 133/12; C11D 17/00; A01N 25/28; C11D 3/386; A61Q 19/00; A61K 8/11; C11D 3/395

(52) U.S. Cl. CPC C09D 133/12; A61K 8/11; C11D 17/0039; C11D 3/395; C11D 3/38672; A61Q 19/00; A01N 25/28

(57) ABSTRACT

The present invention relates to a process for producing a microcapsule dispersion comprising microcapsules comprising a hydrophilic capsule core and a capsule wall polymer, wherein a water-in-oil emulsion comprising a hydrophobic diluent as continuous phase, and the hydrophilic capsule core material, a monomer composition and an amphiphilic polymer is produced and then the monomers are free-radically polymerized,

where the monomer composition comprises

30 to 100% by weight of one or more monomers selected from C₁-C₂₄-alkyl esters of acrylic acid and/or methacrylic acid (monomers I),

0 to 70% by weight of one or more monomers selected from acrylic acid, methacrylic acid, maleic acid, acrylic acid esters and/or methacrylic acid esters which carry hydroxy and/or carboxy groups (monomers II),

0 to 50% by weight of one or more monomers which has two or more ethylenically unsaturated radicals, (monomers III) and

0 to 30% by weight of one or more other monomers (monomers IV)

in each case based on the total weight of the monomers, and the amphiphilic polymer is obtainable by free-radical polymerization of a monomer composition comprising at least one ethylenically unsaturated hydrophilic monomer and at least one ethylenically unsaturated hydrophobic monomer, to the microcapsules obtainable thereby, and to their use for the delayed release of active ingredients for construction, cosmetics, detergents and cleaners or crop protection applications.

The present invention relates to a process for producing a microcapsule dispersion comprising microcapsules comprising a hydrophilic capsule core and a capsule wall polymer, wherein a water-in-oil emulsion comprising a hydrophobic diluent as continuous phase, and the hydrophilic capsule core material, a monomer composition and an amphiphilic polymer is produced and then the monomers are free-radically polymerized,

where the monomer composition comprises
30 to 100% by weight of one or more monomers selected from C₁-C₂₄-alkyl esters of acrylic acid and/or methacrylic acid (monomers I),
0 to 70% by weight of one or more monomers selected from acrylic acid, methacrylic acid, maleic acid, acrylic acid esters and/or methacrylic acid esters which carry hydroxy and/or carboxy groups (monomers II),
0 to 50% by weight of one or more monomers which has two or more ethylenically unsaturated radicals, (monomers III) and
0 to 30% by weight of one or more other monomers (monomers IV)
in each case based on the total weight of the monomers, and the amphiphilic polymer is obtainable by free-radical polymerization of a monomer composition comprising at least one ethylenically unsaturated hydrophilic monomer and at least one ethylenically unsaturated hydrophobic monomer.

Furthermore, the present invention relates to the microcapsules obtainable thereby, and to their use for the delayed release of active ingredients for construction, cosmetics, detergents and cleaners or crop protection applications.

Microcapsules with a hydrophobic capsule core are known for numerous applications. EP 457 154 teaches microcapsules with a color former-comprising core oil and walls which are obtained by polymerization of methacrylates in an oil-in-water emulsion. EP 1 029 018 describes microcapsules with capsule wall polymers based on (meth)acrylates and a capsule core of lipophilic waxes as latent heat storage materials.

Furthermore, WO 2011/064312 teaches microcapsules with crop protection active ingredients dissolved in a hydrophobic oil as capsule core and likewise a capsule wall based on (meth)acrylate.

In contrast to the oil-in-water emulsions in which the oil is the disperse phase, i.e. the discontinuous phase, and the water is the continuous phase, encapsulation processes are also known in which the two phases are swapped. These processes are also referred to as inverse microencapsulation.

DE 10120480 describes such an inverse encapsulation. It teaches microcapsules with a capsule core comprising water-soluble substances and a capsule wall made of melamine/formaldehyde resins. Furthermore, WO 03/015910 teaches microcapsules with a capsule core comprising water-soluble substances and a capsule wall made of polyureas.

EP-A-0 148 169 describes microcapsules with a water-soluble core and a polyurethane wall which are produced in a vegetable oil. The capsule core material specified is, besides herbicides, inter alia water-soluble dyes.

However, there continues to be a need for microcapsules with a water-comprising capsule core which can be used for example as pore formers in construction materials. It is also desired to protect acid in this way whose release can be controlled as accelerator for for example chipboards. The delayed release of water-soluble active ingredients for crop protection or cosmetics applications is also of interest.

The earlier PCT application PCT/EP2012/073932 teaches the production of microcapsules with a hydrophilic capsule core whose capsule wall is a copolymer of (meth)acrylates and hydrophilic (meth)acrylates with hydroxy and/or carboxy groups. The water-in-oil emulsion is stabilized by means of an emulsifier mixture comprising a linear block copolymer with hydrophobic and hydrophilic structural units.

It was the object of the present invention to develop a further process for producing microcapsule dispersions comprising aqueous solutions or else water in the capsule core.

Accordingly, the process described above for producing microcapsules with a hydrophilic capsule core has been found, as have the microcapsules obtainable thereby and their use.

The microcapsules according to the invention comprise a capsule core and a capsule wall. The capsule core consists predominantly, to more than 90% by weight, of water or aqueous solutions. The average particle size D[4,3] of the microcapsules (volume-weighted average, determined by means of laser diffraction) is 0.5 to 100 μm. According to one preferred embodiment, the average particle size of the capsules is 0.5 to 75 μm, preferably 0.5 to 50 μm. Here, preferably 90% of the particles have a particle size of less than twice the average particle size.

The weight ratio of capsule core to capsule wall is generally from 50:50 to 98:2. Preference is given to a core/wall ratio of 70:30 to 95:5.

A hydrophilic capsule core (capsule core material) is to be understood as meaning water and aqueous solutions of water-soluble compounds whose content is at least 10% by weight of a water-soluble compound. Preferably, the aqueous solutions are at least 20% by weight of a water-soluble compound.

The water-soluble compounds are for example organic acids or salts thereof, inorganic acids, inorganic bases, salts of inorganic acids, such as sodium chloride or sodium nitrate, water-soluble dyes, agrochemicals such as Dicamba®, flavorings, pharmaceutical active ingredients, fertilizers or cosmetic active ingredients. Preferred hydrophilic capsule core materials are water and aqueous solutions of organic acids such as acetic acid, formic acid, propionic acid and methanesulfonic acid, and/or salts thereof, inorganic acids such as phosphoric acid and hydrochloric acid, and/or salts of inorganic acids, and sodium silicate.

Depending on the thickness of the capsule wall, which is influenced by the chosen process conditions and also amounts of the feed materials, the capsules are impermeable or sparingly permeable for the hydrophilic capsule core material. With sparingly permeable capsules, a controlled release of the hydrophilic capsule core material can be achieved. The water present in the capsule core will usually evaporate from isolated microcapsules, i.e. microcapsules freed from the hydrophobic diluent, over the course of time.

Wherever (meth is used within the context of this application, both the corresponding acrylates, i.e. the derivatives of acrylic acid, and also the methacrylates, the derivatives of methacrylic acid, are intended.

The polymers of the capsule wall generally comprise at least 30% by weight, in preferred form at least 35% by weight, in particular 40% by weight and in particularly preferred form at least 50% by weight, and also in general at most 100% by weight, preferably at most 95% by weight, in particular at most 90% by weight and in a particularly preferred form at most 85% by weight, of C₁-C₂₄-alkyl esters of acrylic acid and/or methacrylic acid (monomers I) in polymerized-in form, based on the total weight of the monomers.

According to the invention, the polymers of the capsule wall can preferably comprise at least 10% by weight, preferably at least 15% by weight, preferably at least 20% by weight, and in general at most up to 70% by weight, preferably at most 60% by weight, of one or more monomers (II) selected from acrylic acid, methacrylic acid, maleic acid, acrylic acid esters which carry hydroxy and/or carboxy groups, and methacrylic acid esters which carry hydroxy and/or carboxy groups, based on the total weight of the monomers, in polymerized-in form.

In addition, the polymers can preferably comprise at least 5% by weight, preferably at least 10% by weight, preferably at least 15% by weight, and in general at most 50% by weight, preferably at most 40% by weight and in a particularly preferred form at most 30% by weight, of one or more compounds having two or more ethylenically unsaturated radicals (monomers III), in polymerized-in form, based on the total weight of the monomers.

Furthermore, up to 30% by weight of other monomers IV. which are different from monomers I, II and III, may be present in the capsule wall in polymerized-in form.

Preferably, monomer compositions comprising, preferably consisting to at least 95% by weight of, in particular consisting to 100% by weight of,

30 to 100% by weight monomers I,
0 to 70% by weight monomers II
0 to 50% by weight monomers III and
0 to 30% by weight monomers IV
in each case based on the total weight of the monomers, are used for forming the capsule wall.

Suitable monomers I are C₁-C₂₄-alkyl esters of acrylic acid and/or methacrylic acid, and the glycidyl esters of acrylic acid and/or methacrylic acid. Preferred monomers I are methyl, ethyl, n-propyl and n-butyl acrylate, and the corresponding methacrylates. In general, the methacrylates are preferred. Particular preference is given to C₁-C₄-alkyl methacrylates, in particular methyl methacrylate.

According to one particularly preferred embodiment, monomer I is methyl methacrylate and/or one or more C₂-C₂₄-alkyl esters of acrylic acid and/or methacrylic acid. The monomer composition particularly preferably comprises 30-80% by weight of methyl methacrylate.

Monomers II are selected from acrylic acid, methacrylic acid, maleic acid, acrylic acid esters which carry hydroxy and/or carboxy groups, and methacrylic acid esters which carry hydroxy and/or carboxy groups. They are preferably (meth)acrylic acid esters which carry at least one radical selected from carboxylic acid radical and hydroxy radical. The preferred (meth)acrylic acid esters are hydrophilic, i.e. they have a solubility in water of >50 g/l at 20° C. and atmospheric pressure.

The monomers II used are preferably methacrylic acid, hydroxyalkyl acrylates and hydroxyalkyl methacrylates such as 2-hydroxyethyl acrylate and methacrylate, hexapropyl acrylate and methacrylate, hydroxybutyl acrylate and diethylene glycol monoacrylate.

Compounds having two or more ethylenically unsaturated radicals (monomers III) act as crosslinkers. Preference is given to using monomers with vinyl, allyl, acrylic and/or methacrylic groups.

Suitable monomers III having two ethylenically unsaturated radicals are, for example, divinylbenzene and divinylcyclohexane and preferably the diesters of dials with acrylic acid or methacrylic acid, also the diallyl and divinyl ethers of these diols. By way of example, mention may be made of ethanediol diacrylate, ethylene glycol dimethacrylate, 1,3-butylene glycol dimethacrylate, diethylene glycol diacrylate, dipropylene glycol diacrylate, methallylmethacrylamide, allyl acrylate and allyl methacrylate. Particular preference is given to propanediol, butanediol, pentanediol and hexanediol diacrylate and the corresponding methacrylates.

Preferred monomers III having more than two, preferably three, four or more, nonconjugated ethylenic double bonds are the esters of polyalcohols with acrylic acid and/or methacrylic acid, also the allyl and vinyl ethers of these polyalcohols, trivinylbenzene and trivinylcyclohexane. Polyalcohols which may be mentioned here are in particular trimethylol and pentaerythritol. Particular preference is given to trimethylolpropanetriacrylate and -methacrylate, pentaerythritol triallyl ether, pentaerythritol tetraallyl ether, pentaerythritol triacrylate and pentaerythritol tetraacrylate, and their technical-grade mixtures. For example, pentaerythritol tetraacrylate is generally present in technical-grade mixtures in a mixture with pentaerythritol triacrylate and relatively small amounts of oligomerization products.

Suitable other monomers IV are monoethylenically unsaturated monomers which are different from the monomers I and II, such as styrene, β-methylstyrene, β-methylstyrene, vinyl acetate, vinyl propionate and vinyl pyridine.

The water-soluble monomers IV are particularly preferably acrylonitrile, methacrylamide, maleic anhydride, N-vinylpyrrolidone, and acrylamido-2-methylpropanesulfonic acid. In addition, mention is to be made in particular of N-methylolacrylamide, N-methylolmethacrylamide, dimethylaminoethyl methacrylate and diethylaminoethyl methacrylate.

The monomer composition preferably consists of the monomers I and II, and optionally the monomers III and optionally the monomers IV.

Preference is given to using monomer compositions comprising, preferably consisting to at least 95% by weight of, in particular consisting to 100% by weight of

30 to 90% by weight of one or more monomers selected from C₁-C₂₄-alkyl esters of acrylic acid and/or methacrylic acid (monomers I),
10 to 50% by weight of one or more monomers selected from acrylic acid, methacrylic acid, maleic acid, acrylic acid esters and/or methacrylic acid esters which carry hydroxy and/or carboxy groups (monomers II),
0 to 20% by weight of one or more monomers which has two or more ethylenically unsaturated radicals (monomers III)
0 to 10% by weight of one or more other monomers (monomers IV)
in each case based on the total weight of the monomers for the formation of the capsule wall polymer. The monomer composition particularly preferably consists of 55 to 85% by weight of monomers and 15 to 45% by weight of monomers II.

According to a further preferred embodiment, the monomer composition consists of monomers I and III, and optionally monomers II and optionally monomers IV.

Preference is given to using monomer compositions comprising, preferably consisting to at least 95% by weight of, in particular consisting to 100% by weight of

70 to 95% by weight particularly preferably 75 to 90% by weight of one or more monomers selected from C₁-C₂₄-alkyl esters of acrylic acid and/or methacrylic acid (monomers I),
0 to 20% by weight of one or more monomers selected from acrylic acid, methacrylic acid, maleic acid, acrylic acid esters and/or methacrylic acid esters which carry hydroxy and/or carboxy groups (monomers II),
5 to 30% by weight of one or more monomers which has two or more ethylenically unsaturated radicals (monomers III)
0 to 10% by weight of one or more other monomers (monomers IV)
in each case based on the total weight of the monomers.

According to a further preferred embodiment, the monomer composition consists of monomers I, II and III and optionally monomers IV.

Preference is given to using monomer compositions comprising, preferably consisting to at least 95% by weight of, in particular consisting to 100% by weight of

30 to 70% by weight of one or more monomers selected from C₁-C₂₄-alkyl and/or glycidyl esters of acrylic acid and/or methacrylic acid (monomers I),
10 to 50% by weight of one or more monomers selected from acrylic acid, methacrylic acid, maleic acid, acrylic acid esters and/or methacrylic acid esters which carry hydroxy and/or carboxy groups (monomers II)
10 to 50% by weight of one or more monomers which has two or more ethylenically unsaturated radicals (monomers III)
0 to 10% by weight of one or more other monomers (monomers IV)
in each case based on the total weight of the monomers. Preference is given to using a monomer mixture of 30 to 50% by weight of monomers I, 15 to 40% by weight of monomers II, 20 to 50% by weight of monomers III and 0 to 30% by weight of monomers IV for the formation of the capsule wall polymer.

The microcapsules according to the invention are obtainable by preparing a water-in-oil emulsion comprising a hydrophobic diluent as continuous phase, and the hydrophilic capsule core material, the monomers, and the amphiphilic polymer and subsequent free-radical polymerization of the monomers to form the capsule wall polymer. The monomers of the monomer composition can be metered in here in the form of a mixture. However, it is likewise possible to meter them in separately, depending on their hydrophilicity and thus solubility in water or oil, in a mixture with the capsule core material and in a mixture with the hydrophobic diluent. For example, the monomers II are preferably metered in in a mixture with the hydrophilic capsule core material. The monomers I are preferably metered in in a mixture with the hydrophobic diluent.

According to the invention, the continuous phase of the emulsion comprises the amphiphilic polymer in order to avoid a coalescence of the droplets and/or agglomeration of the particles formed. In this emulsion, the water or the aqueous solution is the discontinuous later disperse phase, and the hydrophobic diluent is the continuous phase. The stabilized droplets here have a size which corresponds approximately to the size of the later microcapsules. The wall formation takes place by polymerization of the monomers, which is started by adding a free-radical starter.

Hereinbelow, hydrophobic diluent is understood as meaning diluents which have a solubility in water of <1 g/l, preferably <0.5 g/l at 20° C. and atmospheric pressure. Preferably, the hydrophobic diluent is selected from

cyclohexane,
glycerol ester oils,
hydrocarbon oils, such as paraffin oil, diisopropylnaphthalene, purcellin oil, perhydrosqualene and solutions of microcrystalline waxes in hydrocarbon oils,
animal or vegetable oils,
mineral oils, the distillation start-point of which under atmospheric pressure is ca. 250° C. and the distillation end-point of which is 410° C., such as e.g. Vaseline oil,
esters of saturated or unsaturated fatty acids, such as alkyl myristates, e.g. isopropyl, butyl or cetyl myristate, hexadecyl stearate, ethyl or isopropyl palmitate and cetyl ricinolate,
silicone oils, such as dimethylpolysiloxane, methyl phenyl polysiloxane and the silicon glycol copolymer,
fatty acids and fatty alcohols or waxes such as carnauba wax, candelilla wax, beeswax, microcrystalline wax, ozokerite wax and Ca, Mg and Al oleates, myristates, linoleates and stearates.

Glycerol ester oils are understood as meaning esters of saturated or unsaturated fatty acids with glycerol. Mono-, di- and triglycerides, and their mixtures are suitable. Preference is given to fatty acid triglycerides. Fatty acids which may be mentioned are, for example, C₆-C₁₂-fatty acids such as hexanoic acid, octanoic acid, decanoic acid and dodecanoic acid. Preferred glycerol ester oils are C₆-C₁₂-fatty acid triglycerides, in particular octanoic acid and decanoic acid triglycerides, and their mixtures. Such an octanoyl glyceride/decanoyl glyceride mixture is for example Miglyol® 812 from Hüls.

Particularly preferred hydrophobic diluents are low-boiling alkanes or alkane mixtures such as cyclohexane, naphtha, petroleum, C₁₀-C₁₂-isoalkanes, as are commercially available as Isopar™ G. Furthermore, particular preference is given to using diisopropylnaphthalene, which is available for example as KMC oil from RKS.

In order to obtain a stable emulsion and a homogeneous shell formation, an amphiphilic polymer is used according to the invention that is obtained by free-radical polymerization of a monomer composition comprising ethylenically unsaturated hydrophilic monomers and ethylenically unsaturated hydrophobic monomers. The amphiphilic polymer here preferably exhibits a statistical distribution of the monomer units.

The amphiphilic polymer is preferably positioned, on account of its monomer composition comprising both hydrophilic and hydrophobic units, at the interface of the emulsion droplets and stabilizes these.

Suitable ethylenically unsaturated hydrophobic monomers V comprise long-chain monomers with C₈-C₂₀-alkyl radicals. Of suitability are, for example, alkyl esters of C₈-C₂₀-alcohols, preferably C₁₂- to C₂₀-alcohols, in particular C₁₆-C₂₀-alcohols, with ethylenically unsaturated carboxylic acids, in particular with ethylenically unsaturated C₃-C₆-carboxylic acids such as acrylic acid, methacrylic acid, fumaric acid, itaconic acid and aconitic acid. By way of example, mention may be made of dodecyl acrylate, dodecyl methacrylate, tridecyl acrylate, tridecyl methacrylate, tetradecyl acrylate, tetradecyl methacrylate, octadecyl acrylate, octadecyl methacrylate. Particular preference is given to octadecyl acrylate and octadecyl methacrylate.

Within the context of the ethylenically unsaturated hydrophilic monomers, hydrophilic means that they have a solubility in water of >50 g/l at 20° C. and atmospheric pressure.

Suitable ethylenically unsaturated hydrophilic monomers VI are ethylenically unsaturated monomers with acid groups, and salts thereof, ethylenically unsaturated quaternary compounds, hydroxy (C₁-C₄)alkyl esters of ethylenically unsaturated acids, alkylaminoalkyl (meth)acrylates and alkylaminoalkyl(meth)acrylamides. Ethylenically unsaturated hydrophilic monomers with acid groups or salts of acid groups that may be mentioned by way of example are acrylic acid, methacrylic acid, 2-acrylamide-2-methylpropanesulfonic acid, itaconic acid, maleic acid, fumaric acid. Ethylenically unsaturated quaternary compounds that may be mentioned are dimethylaminoethyl acrylate or methacrylates which are quaternized with methyl chloride. Further suitable ethylenically unsaturated hydrophilic monomers are maleic anhydride and acrylamide.

Besides the ethylenically unsaturated hydrophobic monomers (monomers V) and the ethylenically unsaturated hydrophilic monomers (monomers VI), the amphiphilic polymer can also comprise further comonomers (monomers VII) in polymerized-in form which are different from the monomers of groups V and VI. Ethylenically unsaturated comonomers of this type can be chosen to modify the solubility of the amphiphilic polymer.

Suitable other monomers (monomers VII) are nonionic monomers which optionally have C₁-C₄-alkyl radicals. Preferably, the other monomers are selected from styrene. C₁-C₄-alkylstyrenes such as methylstyrene, vinyl esters of C₃-C6-carboxylic acids such as vinyl acetate, vinyl halides, acrylonitrile, methacrylonitrile, ethylene, butylene, butadiene and other olefins, C₁-C₄-alkyl esters and glycidyl esters of ethylenically unsaturated carboxylic acids. Preference is given to C₁-C₄-alkyl esters and glycidyl esters of ethylenically unsaturated C₃-C₆-carboxylic acids such as acrylic acid, methacrylic acid, fumaric acid, itaconic acid and aconitic acid, for example methyl acrylate, methyl methacrylate, butyl acrylate or butyl methacrylate, and glycidyl methacrylate.

The weight ratio of ethylenically unsaturated hydrophobic monomers/ethylenically unsaturated hydrophilic monomers is preferably 95/5 to 20/80, in particular 90/10 to 30/60.

The amphiphilic polymers comprise in a preferred form at least 20% by weight, particularly preferably at least 30% by weight, in particular 40% by weight and very particularly preferably at least 45% by weight, and preferably at most 95% by weight, preferably at most 90% by weight, of ethylenically unsaturated hydrophobic monomers V in polymerized-in form, based on the total weight of the monomers.

The amphiphilic polymers comprise in a preferred form at least 5% by weight, particularly preferably at least 7% by weight, and very particularly preferably at least 10% by weight, and preferably at most 80% by weight, preferably at most 60% by weight, and particularly preferably at most 50% by weight, of ethylenically unsaturated hydrophilic monomers VI in polymerized-in form, based on the total weight of the monomers.

The amphiphilic polymers comprise in a preferred form at least 5% by weight, particularly preferably at least 7% by weight, in particular 10% by weight, and preferably at most 55% by weight, preferably at most 45% by weight, of other monomers VII in polymerized-in form, based on the total weight of the monomers.

Preference is given to using amphiphilic polymers which are obtainable by free-radical polymerization of a monomer composition comprising, preferably consisting of

20 to 90% by weight of one or more ethylenically unsaturated hydrophobic monomers (monomers V),
5 to 50% by weight of one or more ethylenically unsaturated hydrophilic monomers (monomers VI),
0 to 45% by weight of one or more other monomers (monomers VII)
in each case based on the total weight of the monomers V, VI and VII.

Particular preference is given to choosing amphiphilic polymers which are obtainable by free-radical polymerization of a monomer composition comprising, preferably consisting of

20 to 90% by weight of one or more alkyl esters of C₈-C₂₀-alcohols with ethylenically unsaturated carboxylic acids,
5 to 50% by weight of one or more monomers selected from ethylenically unsaturated monomers with acid groups, and salts thereof, ethylenically unsaturated quaternary compounds, hydroxy (C₁-C₄)alkyl esters of ethylenically unsaturated acids, alkylaminoalkyl (meth)acrylates, alkylaminoalkyl (meth)acrylamides, maleic anhydride and acrylamide,
0 to 45% by weight of one or more monomers selected from styrene. C₁-C₄-alkylstyrenes, vinyl esters of C₃-C₆-carboxylic acids, vinyl halides, acrylonitrile, methacrylonitrile, ethylene, butylenes, butadiene and C₁-C₄-alkyl esters of ethylenically unsaturated C₃-C₆-carboxylic acids in each case based on the total weight of the monomers.

Particular preference is given to amphiphilic polymers which are obtainable by free-radical polymerization of a monomer composition comprising, preferably consisting of:

40 to 90% by weight of one or more alkyl esters of C₁₆-C₂₀-alcohols with ethylenically unsaturated carboxylic acids,
10 to 35% by weight of one or more monomers selected from acrylic acid, methacrylic acid, 2-acrylamido-2-methylpropanesulfonic acid, itaconic acid, maleic acid, fumaric acid, maleic anhydride and acrylamide,
0 to 40% by weight of one or more monomers selected from styrene, C₁-C₄-alkylstyrenes, vinyl esters of C₃-C₆-carboxylic acids, vinyl halides, acrylonitrile, methacrylonitrile and methyl methacrylate
in each case based on the total weight of the monomers.

Furthermore, preference is given to amphiphilic polymers which are obtainable by free-radical polymerization of a monomer composition comprising, preferably consisting of,

60 to 90% by weight of one or more alkyl esters of C₁₆-C₂₀-alcohols with ethylenically unsaturated carboxylic acids,
10 to 35% by weight of one or more monomers selected from acrylic acid, methacrylic acid, 2-acrylamido-2-methylpropanesulfonic acid, itaconic acid, maleic acid, fumaric acid, maleic anhydride and acrylamide,
0 to 10% by weight of one or more monomers selected from styrene, C₁-C₄-alkylstyrenes, vinyl esters of C₃-C₆-carboxylic acids, vinyl halides, acrylonitrile, methacrylonitrile and methyl methacrylate
in each case based on the total weight of the monomers.

Furthermore, preference is given to amphiphilic polymers which are obtainable by free-radical polymerization of a monomer composition comprising, preferably consisting of,

40 to 70% by weight of one or more alkyl esters of C₁₆-C₂₀-alcohols with ethylenically unsaturated carboxylic acids,
10 to 35% by weight of one or more monomers selected from acrylic acid, methacrylic acid, 2-acrylamido-2-methylpropanesulfonic acid, itaconic acid, maleic acid, fumaric acid, maleic anhydride and acrylamide,
10 to 40% by weight of one or more monomers selected from styrene. C₁-C₄-alkylstyrenes, vinyl esters of C₃-C₆-carboxylic acids, vinyl halides, acrylonitrile, methacrylonitrile and methyl methacrylate
in each case based on the total weight of the monomers.

The amphiphilic polymer generally has an average molecular weight M_{w} (determined by means of gel permeation chromatography) of from 5000 to 500 000, preferably from ≧10 000 up to 400 000 and particularly preferably 30 000 to 200 000.

The amphiphilic polymers are preferably prepared by initially introducing the total amount of the monomers in the form of a mixture and then carrying out the polymerization. Furthermore, it is possible to meter in the monomers under polymerization conditions discontinuously in one or more part amounts or continuously in constant or changing quantitative streams.

The optimum amount of amphiphilic polymer for stabilizing the hydrophilic droplets before the reaction and the microcapsules after the reaction is influenced firstly by the amphiphilic polymer itself, secondly by the reaction temperature, the desired microcapsule size and by the wall materials, and also the core composition. The optimally required amount can be ascertained easily by simple experimental series. As a rule, the amphiphilic polymer is used for preparing the emulsion in an amount of from 0.01 to 15% by weight, preferably 0.05 to 12% by weight and in particular 0.1 to 10% by weight, based on the capsules (wall and core).

Polymerization initiators that can be used are all compounds that disintegrate into free radicals under the polymerization conditions, e.g. peroxides, hydroperoxides, persulfates, azo compounds and the so-called redox initiators.

In some cases, it is advantageous to use mixtures of different polymerization initiators, e.g. mixtures of hydrogen peroxide and sodium or potassium peroxodisulfate. Mixtures of hydrogen peroxide and sodium peroxodisulfate can be used in any desired ratio. Suitable organic peroxides are for example acetylacetone peroxide, rnethylethyl ketone peroxide, tert-butyl hydroperoxide, cumene hydroperoxide, tert-amyl perpivalate, tert-butyl perpivalate, tert-butyl perneohexanoate, tert-butyl perisobutyrate, tert-butylper-2-ethylhexanoate, tert-butyl perisononanoate, tert-butyl permaleate, tert-butyl perbenzoate, tert-butyl per-3,5,5-tri-methylhexanoate and tert-amyl perneodecanoate. Further suitable polymerization initiators are azo starters, e.g. 2,2′-azobis(2-amidinopropane) dihydrochloride, 2,2′-azobis(N,N-dimethylene)isobutyramidine dihydrochloride, 2-(carbamoylazo)isobutyronitrile and 4,4′-azobis(4-cyanovaleric acid).

Preference is given to using azo starters and peroxides as polymerization initiators. The specified polymerization initiators are used in customary amounts, e.g. in amounts of from 0.1 to 5, preferably 0.1 to 2.5 mol %, based on the monomers to be polymerized.

The dispersing of the core material takes place in a known manner depending on the size of the capsules to be produced. For producing large capsules, dispersion using effective stirrers suffices, in particular anchor and MIG (cross-blade) stirrers. Small capsules, particularly if the size is to be less than 20 μm, require homogenization or dispersing machines.

The capsule size can be controlled within certain limits via the rotational speed of the dispersing device/homogenizing device and/or with the help of the concentration of the amphiphilic polymer and/or via its molecular weight, i.e. via the viscosity of the continuous phase. In this connection, the size of the dispersed droplets decreases as the rotational speed increases up to a limiting rotational speed.

In this connection, it is important that the dispersing devices are used at the start of capsule formation. For continuously operating devices with forced throughflow, it is sometimes advantageous to send the emulsion through the shear field several times.

As a rule, the polymerization is carried out at 20 to 100° C., preferably at 40 to 95° C. Expediently, the polymerization is performed at atmospheric pressure, although it is also possible to work at reduced or slightly increased pressure, e.g. at a polymerization temperature above 100° C., thus for example in the range from 0.5 to 5 bar.

The reaction times of the polymerization are normally 1 to 10 hours, mostly 2 to 5 hours.

Microcapsule dispersions with a content of from 5 to 50% by weight of microcapsules can be produced by the process according to the invention. The microcapsules are individual capsules. Capsules with an average particle size in the range from 0.5 up to 100 μm can be produced by selecting suitable conditions during dispersion. Preference is given to capsules with an average particle size of from 0.5 to 75 μm, in particular up to 50 μm.

Depending on the field of use, it may be advantageous to use the microcapsules directly as microcapsule dispersion as obtained according to the process above. Furthermore, it may be advantageous to use the microcapsules in the form of a solid.

The microcapsules obtained can be isolated by removing the hydrophobic solvent. This can be performed for example by evaporating off the hydrophobic solvent or by means of suitable spray-drying processes in an inert gas atmosphere.

The process according to the invention permits the production of microcapsules with a hydrophilic capsule core and a capsule wall made of a polymer based on (meth)acrylic acid esters. The capsules according to the invention can be used in a very wide variety of fields depending on the core material. In this way, it is possible to convert hydrophilic liquids or mixtures of organic acids or salts thereof, inorganic acids, inorganic bases, salts of inorganic acids, water-soluble dyes, flavorings, pharmaceutical active ingredients, fertilizers, crop protection active ingredients, active ingredients for detergents and cleaners, for example enzymes, or cosmetic active ingredients to a solid formulation or oil-dispersible formation which releases these as required.

For example, microcapsules with a water core are suitable as pore formers for concrete. A further application in construction materials is the use of encapsulated water-soluble catalysts in binding construction materials.

Microcapsules with encapsulated inorganic or organic acids can be used advantageously as boring aids for, for example, geothermal wells since they permit release only at the drilling site. Thus, they allow the increase in permeability in subterranean, carbonatic petroleum- and/or natural-gas-bearing and/or hydrothermal rock formations. For example, these capsules can be used for dissolving carbonatic and/or carbonate-containing impurities during the recovery of petroleum and/or natural gas or the recovery of energy by hydrothermal geothermy by forcing a formulation comprising microcapsules according to the invention with encapsulated inorganic or organic acids into the rock formation through at least one borehole. Furthermore, encapsulated acids, which permit a delayed or targeted release of the acid, are also suitable as catalysts for producing chipboards.

Furthermore, the microcapsule dispersion according to the invention with water-soluble bleaches or enzymes as core material permits use as a constituent in detergents and cleaners, especially in liquid formulations. Bleaches of this type are generally based on organic and/or inorganic peroxygen compounds. Consequently, the present invention also relates to the use of the microcapsule dispersion in detergents for textiles and in cleaners for nontextile surfaces. Besides the microcapsules according to the invention, such detergents and cleaners can comprise builder substances, surface-active surfactants, bleaches, bleach activators, water-miscible organic solvents, enzymes, sequestrants, electrolytes, pH regulators and further auxiliaries, such as optical brighteners, graying inhibitors, foam regulators, and dyes and fragrances.

Furthermore, active ingredients which are to be released in a controlled manner, be they medicinal active ingredients, cosmetic active ingredients or crop protection active ingredients, can be prepared in such a manner since release takes place over a prolonged period as a function of the denseness of the capsule wall.

### EXAMPLES

Preparation of the Amphiphilic Polymers

Amphiphilic polymer solution S1

Initial charge:

280.00 g Isopar™ G (low-boiling alkane mixture with a density of 0.75 g/cm³ at 20° C., ExxonMobil)
23.10 g Stearyl methacrylate

Feed 1:

### 532.00 g Isopar™ G

92.40 g Stearyl methacrylate
69.30 g Methyl methacrylate
4.20 g Glycidyl methacrylate
21.00 g Methacrylic acid

Feed 2:

1.68 g 2,2′-azobis(2-methylbutyronitrile) (Wako V 59)

### 3.38 g Toluene

### 50.96 g Isopar™ G

The initial charge was introduced and heated to 85° C. Feed 2 was then started. After 5 minutes, feed 1 was started and both feeds were metered in over 2 hours. The temperature was then held at 85° C. for 2 hours and then the mixture was cooled to room temperature. This gave a solution of the polymer in Isopar™ G with a solids content of 19.6% by weight. The polymer has a molecular weight Mn of 34 730 g/mol and—

Mw=172 100 g/mol.

Furthermore, the following amphiphilic polymer solutions, which were prepared analogously to amphiphilic polymer solution S1, were used:

Amphiphilic polymer solution S2: polymer of 65 equivalents by weight stearyl methacrylate, 17.5 equivalents by weight maleic anhydride and 17.5 equivalents by weight of styrene, in the form of a 35.0% strength by weight solution in Isopar™G/white oil (2:1).

Amphiphilic polymer solution S3: polymer of 88 equivalents by weight stearyl methacrylate and 12 equivalents by weight methacrylic acid, in the form of a 31.0% strength by weight solution in Isopar™ G.

Amphiphilic polymer solution S4: polymer based on 66.7 equivalents by weight stearyl methacrylate and 33.3 equivalents by weight of methacrylic acid, in the form of a 22.2% strength by weight solution in aliphatic hydrocarbons.

Amphiphilic polymer S5: polymer of stearyl methacrylate and methyl methacrylate, in the form of a 25% strength by weight solution in Isopar™ G.

Amphiphilic polymer S6: polymer of 39.5 equivalents by weight methyl methacrylate, 48.1 equivalents by weight stearyl methacrylate, 6.2 equivalents by weight methacrylic acid and 6.2 equivalents by weight acrylic acid, in the form of a 30.8% strength by weight solution in Isopar G.

Gel Permeation Chromatography

The molar mass distribution of the amphiphilic polymer was determined by size exclusion chromatography (SEC). The elution curve was converted to the actual distribution curve with the help of a polystyrene calibration curve (polystyrene standard (580 g/mol to 7 500 000 g/mol) from Polymer Laboratories GmbH) and with calibration by means of hexylbenzene (162 g/mol). The eluent was tetrahydrofuran admixed with 0.1% by weight of trifluoroacetic acid. The injection volume was 100 μl with a flow rate of 1 ml/g. The sample concentration was 2 mg/ml and the column temperature 35° C. A set of 3 columns from Agilent Technologies was used:

1st column: L=50 mm, ID=7.5 mm, Agilent PLgel 10 μm Guard (precolumn)
2nd column: L=300 mm, ID=7.5 mm, Agilent PLgel 10 μm MIXED-B
3rd column: L=300 mm, ID=7.5 mm, Agilent PLgel 10 μm MIXED-B

Preparation of oil-based microcapsule dispersions

### Example 1

Oil Phase:

### 356.87 g Isopar G

45.05 g of the amphiphilic polymer solution S4

Feed 1:

200.00 g Demin. water

Feed 2:

40.00 g Methyl methacrylate
10.00 g 1,4-Butanediol diacrylate

Feed 3:

1.33 g tert-Butyl perpivalate

Feed 4:

0.50 g tert-Butyl peroxyneodecanoate

The oil phase was introduced and feeds 1 and 2 were added. The mixture was emulsified for 40 minutes at 3500 rpm. Then, feed 3 was added and the mixture was heated to a temperature of 75° C. over a period of 10 minutes. The mixture was held at this temperature for 1 hour and then heated up to 85° C. in 10 minutes and held at this temperature for a further 2 hours. Then, over the course of 1 hour, the mixture was cooled to room temperature and during this time feed 4 was added. The wall thickness of the microcapsules was 20% by weight, based on wall and core. The solids content was 40% by weight.

### Example 2

Oil phase:

### 426.57 g Diisopropylnaphthalene

66.67 g of the amphiphilic polymer solution S1

Feed 1:

222.75 g Demin. water
2.25 g Sodium chloride

Feed 2:

21.25 g Methyl methacrylate
2.50 g 1,4-Butanediol diacrylate
1.25 g 2-Hydroxyethyl methacrylate

Feed 3:

0.33 g tert-Butyl perpivalate

The oil phase was introduced and feed 1 was added. The mixture was emulsified for 40 minutes at 4000 rpm. It was then heated to 85° C. and feed 3 was added. Feed 2 was metered in over 1 hour and the mixture was then held at this temperature for a further 2 hours. The mixture was then cooled to room temperature in 1 hour. This gave an oil-based microcapsule dispersion with an average particle size of D[4,3]=34.2 μm. The wall thickness of the microcapsules was 10% by weight, based on wall and core. The solids content was 35% by weight.

### Example 3

Analogously to example 2, 225 g of water were encapsulated without sodium chloride. This gave an oil-based microcapsule dispersion with an average particle size of D[4,3]=78.3 μm. The wall thickness of the microcapsules was 10% by weight, based on wall and core. The solids content was 35% by weight.

### Example 4

Oil phase:

### 426.57 g Diisopropylnaphthalene

33.33 g of the amphiphilic polymer solution S6

Feed 1:

202.50 g Demin. water
22.50 g Sodium chloride

### 0.50 g Basacid Blau 756 (BASF) (C.I. 42090 Acid Blue 9)

Feed 2:

2125 g Methyl methacrylate
2.50 g 1,4-Butanediol diacrylate
1.25 g 2-Hydroxyethyl methacrylate

Feed 3:

0.33 g tert-Butyl perpivalate

The oil phase was introduced and heated to 85° C., and feed 1 was added. The mixture was emulsified for 40 minutes at 4000 rpm. Then, feed 3 was added. Feed 2 was metered in over 1 hour and the mixture was then held at this temperature for a further 2 hours. The mixture was then cooled to room temperature over 1 hour. This gave an oil-based microcapsule dispersion with an average particle size of D[4,3] =45.9 μm. The wall thickness of the microcapsules was 10% by weight, based on wall and core. The solids content was 36.6% by weight of an average particle size of D[4,3]=45.9 μm. The wall thickness of the microcapsules was 10% by weight, based on wall and core.

### Example 5

Oil phase:

### 617.52 g Cyclohexane

60.00 g of the amphiphilic polymer solution S6

Feed 1:

380.00 g Demin. water

Feed 2:

8.00 g Methyl methacrylate
4.00 g 1,4-Butanediol diacrylate
8.00 g Methacrylic acid

Feed 3:

### 0.20 g Wako V 59

### 100.00 g Cyclohexane

The oil phase was introduced, feed 1 was added and the mixture was emulsified for 20 minutes at 3500 rpm. The mixture was then heated to 75° C. and feed 2 was introduced over 2 hours, and feed 3 was introduced over 2.5 hours. Then, the temperature was held at 75° C. for a further 60 minutes. This gave an oil-based microcapsule dispersion with a solids content of 35.51%. The cyclohexane was then distilled off and cooled to room temperature.

### Example 6

Example 6 was performed analogously to example 5, with 4.00 g of 1,4-butanediol diacrylate being replaced by 4.00 g of pentaerythritol triacrylate.

### Example 7

Oil phase:

### 617.52 g Cyclohexane

40.00 g of the amphiphilic polymer solution S6

Feed 1:

360.00 g Demin. water

Feed 2:

16.00 g Methyl methacrylate
8.00 g Stearyl methacrylate
16.00 g Methacrylic acid

Feed 3:

### 0.20 g Wako V 59

### 100.00 g Cyclohexane

The oil phase was introduced, feed 1 was added and the mixture was emulsified for 20 minutes at 3500 rpm. The mixture was then heated to 75° C. and feed 2 was introduced over 2 hours and feed 3 was introduced over 2.5 hours. The temperature was then held at 75° C. for a further 60 minutes. This gave an oil-based microcapsule dispersion with a solids content of 35.6%. The cyclohexane was then distilled off and cooled to room temperature.

Non-inventive example 8:

Oil phase:

### 484.16 g Diisopropylnaphthalene

### 10.00 g Tamol® DN (BASF)

Feed 1:

202.50 g Demin. water
22.50 g Sodium chloride

### 0.50 g Basacid Blau 756 (BASF) (CA. 42090 Acid Blue 9)

Feed 2:

21.25 g Methyl methacrylate
2.50 g 1,4-Butanediol diacrylate
1.25 g 2-Hydroxyethyl methacrylate

Feed 3:

0.33 g tert-Butyl perpivalate

The procedure was as in example 4 except that the emulsifier used was Tamol® DN (anionic surfactant: sodium salt of condensation product of naphthalenesulfonic acid). The oil phase was introduced and heated to 85° C., and feed 1 was added. Emulsification was carried out for 40 minutes at 4000 rpm. Feed 3 was then added. Feed 2 was metered in over 1 hour and then the mixture was held at this temperature for a further 2 hours. Cooling to room temperature was then carried out over 1 hour. This gave an oil-based microcapsule dispersion with an average particle size of D[4,3] =153.4 μm. The wall thickness of the microcapsules was 10% by weight, based on wall and core. The solids content was 35% by weight.

TABLE 1
Overview of the oil-based microcapsule dispersions obtained

| | | | | Solids con- |
|---|---|---|---|---|
| | | | Amphi- | tent of the |
| Exam- | Core | | philic | dispersion |
| ple | material | Wall composition | polymer | [% by weight] |
| 1 | Water | MMA/BDDA | S4 | 40 |
| 2 | Water/NaCl | MMA/BDDA/HEMA | S1 | 35 |
| 3 | Water | MMA/BDDA/HEMA | S1 | 35 |
| 4 | Water/NaCl/ | MMA/BDDA/HEMA | S6 | 36.7 |
| | Basacid | | | |
| | Blau 756 | | | |
| 5 | Water | MMA/BDDA/MAA | S6 | 35.5 |
| 6 | Water | MMA/PETIA/MAA | S6 | 35.5 |
| 7 | Water | MMA/SMA/MAA | S6 | 35.6 |
| 8 | Water/NaCl/ | MMA/BDDA/HEMA | Tamol | 35 |
| | Basacid | | DN | |
| | Blau 756 | | | |

MMA: Methyl methacrylate
BDDA: 1,4-Butanediol diacrylate
HEMA: 2-Hydroxyethyl methacrylate
SMDA: Stearyl methacrylate
PETIA: Pentaerythritol triacrylate
MAA: Methacrylic acid
Release experiments

To test the capsule quality, comparative release experiments on the dispersion from example 4 and example 8 were carried out.

Measurement: the dye Basacid Blau 756 in the capsule core is exclusively water-soluble and cannot be detected in the continuous oil phase. A calibration curve was drawn up by preparing aqueous solutions of this dye of varying concentration β (0.00051 g/l to 0.01303 g/l) and their extinction E was measured at 630 nm using a UVNIS spectrometer (UV1800 Shimadzu) in single-use cuvettes 1 cm in thickness (polystyrene, VWR):

TABLE 3
Determination of the calibration curve for aqueous Basacid
Blau 756 solutions: β (g/l) = concentration of
Basacid Blau 756 in aqueous solution, E = measured extinction.

| β (g/l) | E |
|---|---|
| 0.000507 | 0.0567 |
| 0.003051 | 0.3502 |
| 0.005088 | 0.5856 |
| 0.0086951 | 1.0006 |
| 0.013027 | 1.4942 |

By reference to the linear curve, it is possible to determine the dye concentration β (g/l) for a measured extinction (E=114.84 (l/g)*β(g/l)).

To investigate the release of the dye from the microcapsules, the capsule dispersions were treated as follows: the mass of ca. 1 g of the dispersion was weighed out and topped up to 100 ml with a 10% strength sodium dodecylsulfate solution (surfactant solution) and stirred for 24 hours on a magnetic stirrer. The surfactant solution solubilized the released dye. Then, a sample was taken from this mixture and filtered through a 0.45 μm syringe filter in order to separate the solution, comprising the released dye, from the microcapsules. The filtrate was measured in the UV-VIS spectrometer (UV1800, Shimadzu) at 630 nm in 1 cm single-use cuvettes (polystyrene. VV/R) and the extinction was determined. With the help of the linear calibration curve determined within this concentration range (E=114.84 (l/g)*β(g/l)) and the known amount of dye used, it is possible to determine the percentage release of the dye (see table 3).

TABLE 3
Results of the release measurements

| | | | Concentration | Percentage |
|---|---|---|---|---|
| | Masses of | | of released | released |
| Capsule | dispersion | Measured | Basacid Blau | Basacid |
| dispersion | used | extinction E | 756 [g/l] | Blau 756 |
| Example 4 | 0.991 g | 0.2893 | 0.002519 | 35.8% |
| Example 8 (not | 1.005 g | 0.7426 | 0.006466 | 94.2% |
| inventive) | | | | |

It is evident from table 3 that using the amphiphilic polymer according to the invention leads to a considerably reduced release of the dye and thus to tighter microcapsules according to the invention compared to using an ionic surfactant as emulsifier.

1-17. (canceled)

18. A process for producing a microcapsule dispersion comprising microcapsules comprising a hydrophilic capsule core and a capsule wall polymer, wherein a water-in-oil emulsion comprising a hydrophobic diluent as continuous phase, and the hydrophilic capsule core material, a monomer composition and an amphiphilic polymer is produced and then the monomers are free-radically polymerized,
where the monomer composition comprises
30 to 100% by weight of one or more monomers selected from C₁-C₂₄-alkyl esters of acrylic acid and/or methacrylic acid,
0 to 70% by weight of one or more monomers selected from acrylic acid, methacrylic acid, maleic acid, acrylic acid esters and/or methacrylic acid esters which carry hydroxy and/or carboxy groups,
0 to 50% by weight of one or more monomers which has two or more ethylenically unsaturated radicals, and
0 to 30% by weight of one or more other monomers
in each case based on the total weight of the monomers, and the amphiphilic polymer is obtainable by free-radical polymerization of a monomer composition comprising at least one ethylenically unsaturated hydrophilic monomer and at least one ethylenically unsaturated hydrophobic monomer.

19. The process according to claim 18, wherein the hydrophilic capsule core of the microcapsules is selected from water and aqueous solutions of organic acids, and salts thereof, inorganic acids, and inorganic salts and sodium silicate.

20. The process according to claim 18, wherein the monomer composition comprises methyl methacrylate.

21. The process according to claim 18, wherein the monomer composition comprises
30 to 90% by weight of one or more monomers selected from C₁-C₂₄-alkyl esters of acrylic acid and/or methacrylic acid,
10 to 50% by weight of one or more monomers selected from acrylic acid, methacrylic acid, maleic acid, acrylic acid esters and/or methacrylic acid esters which carry hydroxy and/or carboxy groups,
0 to 20% by weight of one or more monomers which has two or more ethylenically unsaturated radicals,
0 to 10% by weight of one or more other monomers
in each case based on the total weight of the monomers.

22. The process according to claim 18, wherein the monomer composition comprises
70 to 95% by weight of one or more monomers selected from C₁-C₂₄-alkyl esters of acrylic acid and/or methacrylic acid,
0 to 20% by weight of one or more monomers selected from acrylic acid, methacrylic acid, maleic acid, acrylic acid esters and/or methacrylic acid esters which carry hydroxy and/or carboxy groups,
5 to 30% by weight of one or more monomers which has two or more ethylenically unsaturated radicals,
0 to 10% by weight of one or more other monomers
in each case based on the total weight of the monomers.

23. The process according to claim 22, wherein the monomer composition comprises 75 to 90% by weight of one or more monomers selected from C₁-C₂₄-alkyl esters of acrylic acid and/or methacrylic acid, based on the total weight of the monomers.

24. The process according to claim 18, wherein the monomer composition comprises
30 to 70% by weight of one or more monomers selected from C₁-C₂₄-alkyl and/or glycidyl esters of acrylic acid and/or methacrylic acid,
15 to 50% by weight of one or more monomers selected from acrylic acid, methacrylic acid, maleic acid, acrylic acid esters and/or methacrylic acid esters which carry hydroxy and/or carboxy groups,
10 to 50% by weight of one or more monomers which has two or more ethylenically unsaturated radicals and
0 to 10% by weight of one or more other monomers
in each case based on the total weight of the monomers.

25. The process according to claim 18, wherein the amphiphilic polymer is obtainable by free-radical polymerization of a monomer composition comprising
20 to 90% by weight of one or more ethylenically unsaturated hydrophobic monomers (monomers V),
5 to 50% by weight of one or more ethylenically unsaturated hydrophilic monomers (monomers VI),
0 to 45% by weight of one or more other monomers (monomers VII)
in each case based on the total weight of the monomers V, VI and VII.

26. The process according to claim 18, wherein the amphiphilic polymer is obtainable by free-radical polymerization of a monomer composition comprising
20 to 90% by weight of one or more alkyl esters of C₈-C₂₀-alcohols with ethylenically unsaturated carboxylic acids,
5 to 50% by weight of one or more monomers selected from ethylenically unsaturated monomers with acid groups, and salts thereof, ethylenically unsaturated quaternary compounds, hydroxy (C₁-C₄)alkyl esters of ethylenically unsaturated acids, alkylaminoalkyl (meth)acrylates, alkylaminoalkyl(meth)acrylamides, maleic anhydride and acrylamide,
0 to 45% by weight of one or more monomers selected from styrene, C₁-C₄-alkylstyrenes, vinyl esters of C₃-C₆-carboxylic acids, vinyl halides, acrylonitrile, methacrylonitrile, ethylene, butylenes, butadiene and C₁-C₄-alkyl esters of ethylenically unsaturated C₃-C₆-carboxylic acids
in each case based on the total weight of the monomers.

27. The process according to claim 18, wherein the amphiphilic polymer has an average molecular weight M_{w} of from 5000 to 500 000.

28. The process according to claim 18, wherein the hydrophobic diluent has a solubility in water <0.5 g/l at 20° C. and atmospheric pressure.

29. The process according to claim 18, wherein the microcapsules obtained are isolated by removing the hydrophobic solvent.

30. A microcapsule dispersion obtainable according to the process of claim 18.

31. A microcapsule obtainable according to the process of claim 18.

32. As auxiliary for modifying binding construction materials which comprises the microcapsule according to claim 30, comprising water or inorganic acids

33. A cosmetic active ingredient as core material as constituent in cosmetic preparations which comprises the microcapsule according to claim 30.

34. A crop protection active ingredient as core material as constituent in agrochemical formulation which comprises the microcapsule according to claim 30.

35. A water-soluble bleach or enzyme as core material as constituent of a detergent or cleaner which comprises the microcapsule according to claim 30.
